(19) United States
(12) Patent Application Publication
(10) Pub. No.: US 2006/0252940 A1
(43) Pub. Date: Nov. 9, 2006

(54) CRYSTALLINE 1-[2-(2,4-DIFLUOROPHENYL)-OXIRANYL METHYL]-1H-1,2,4-TRIAZOLE

(76) Inventors: Sudhakar Valluri, West Godavari, Andhra Pradesh-534411 (IN); Sandeep Mohanty, Bhubaneswar, Orissa-751020 (IN); Chandra Mohan Reddy Devireddy, Kadapa, Andhra Pradesh -516484 (IN); Srinivasa Rao Talasila, Krishna, Andhra Pradesh-521185 (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/381,549

(22) Filed: May 4, 2006

Related U.S. Application Data
(60) Provisional application No. 60/691,942, filed on Jun. 17, 2005.

(30) Foreign Application Priority Data
May 5, 2005 (IN) 533/CHE/2005

(51) Int. Cl. C07D 405/02

(52) U.S. Cl. 548/268.8

(57) ABSTRACT

A crystalline form of the compound 1-[2-(2,4-difluorophenyl)-oxiranyl methyl]-1H-1,2,4-triazole and a process for preparing the form. The crystalline form can be used for preparing fluconazole.

### INTRODUCTION TO INVENTION

The present invention relates to crystalline 1-[2-(2,4-difluorophenyl)-oxiranyl methyl]-1H-1,2,4-triazole of Formula I, which is useful as an intermediate in a process for the production of fluconazole, and a process for preparation thereof.

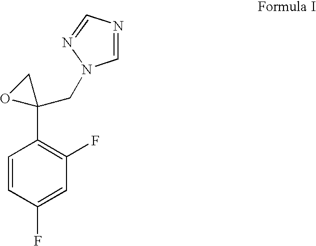

Fluconazole has the chemical name 2-(2,4-difluorophenyl)-1,3-bis-(1H-1,2,4-triazol-1-yl)-1 propanol and can be represented by the structural Formula II.

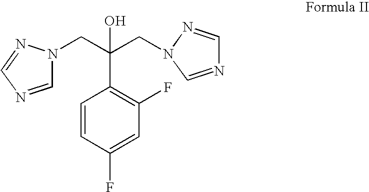

Fluconazole is a synthetic triazole antifungal agent which is commercially available in the market in products sold under the trademark DIFLUCAN. It is available for oral administration as tablets containing 50, 100, 150, or 200 mg of fluconazole.

U.S. Pat. No. 4,404,216 discloses fluconazole and its pharmaceutically acceptable salts. This patent also describes a process for its preparation. Indian Journal of Chemistry, Vol. 42B, February 2003, pp. 372-381 also describes a process for the preparation of fluconazole.

The compound of Formula I described above is a key intermediate in the synthesis of fluconazole. Several methods for the preparation of this compound are reported in literature, but all the methods yield an unstable oily product.

The preparation of fluconazole entails numerous steps. An example of such preparative multi-step synthesis is the process described in U.S. Pat. No. 4,404,216. In such synthesis, it is desirable to obtain the intermediates in the individual steps in highly purified form for use in the succeeding steps. Crystallinity of intermediates reflects their purity and is highly desirable since unwanted side reactions involving impurities can be avoided in the subsequent steps of the overall process.

The present invention provides a crystalline form of the intermediate of Formula I, and its isolation which affords an additional purification not possible in the prior art processes.

The process of the present invention has advantages of improved yield and increased productivity which affords a significantly greater weight of the product fluconazole of Formula II. The process is also industrially scaleable, and cost effective.

### SUMMARY OF THE INVENTION

The present invention relates to a crystalline form of the intermediate 1-[2-(2,4-difluorophenyl)-oxiranyl methyl]-1H-1,2,4-triazole of Formula I, and a process for preparation thereof.

In an embodiment, the process for the preparation of crystalline 1-[2-(2,4-difluorophenyl)-oxiranyl methyl]-1H-1,2,4-triazole of Formula I comprises the steps of:

a) Reacting 2,4-difluoro-2-(1H-1,2,-4-trazol-1-yl)acetophenone of Formula III with trimethyl sulphoxonium iodide in the presence of a phase transfer catalyst and a base in a suitable solvent.

b) Isolation of crystalline 1-[2-(2,4-difluorophenyl)-oxiranyl methyl]-1H-1,2,4-triazole of Formula I from the reaction mass of step a).

Another aspect of the invention involves a process for preparing fluconazole of Formula II using the crystalline intermediate of Formula I.

In an embodiment, a process for the preparation of fluconazole of Formula II from the crystalline intermediate of Formula I comprises reacting the crystalline intermediate of Formula I with 1,2,4-triazole in the presence of a base in a suitable solvent.

### BRIEF DESCRIPTION OF THE DRAWINGS

FIG. 1 is a schematic representation of a process for the preparation of fluconazole.

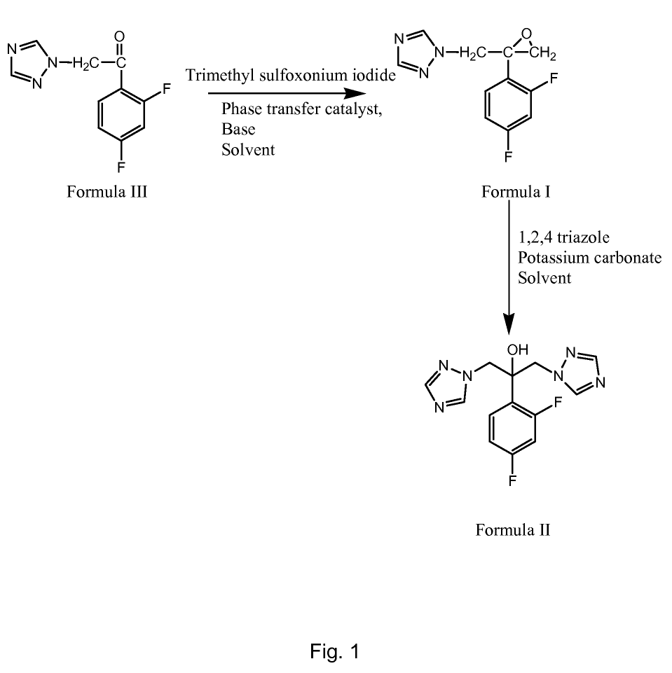

FIG. 2 is an X-ray powder diffraction pattern for 1-[2-(2,4-difluorophenyl)-oxiranyl methyl]-1H-1,2,4-triazole of Formula I, as prepared in Example 1.

### DETAILED DESCRIPTION OF THE INVENTION

In an aspect, the present invention relates to a crystalline 1-[2-(2,4-difluorophenyl)-oxiranyl methyl]-1H-1,2,4-triazole of Formula I, and a process for preparation thereof.

Crystalline 1-[2-(2,4-difluorophenyl)-oxiranyl methyl]-1H-1,2,4-triazole of Formula I obtained in the present invention is characterized by its X-ray powder diffraction pattern (“XRPD”) pattern. The XRPD data reported herein were obtained using Cu Kα-1 radiation, having the wavelength 1.541 Å, and were obtained using a Bruker Axe D8 Advance Powder X-ray Diffractometer.

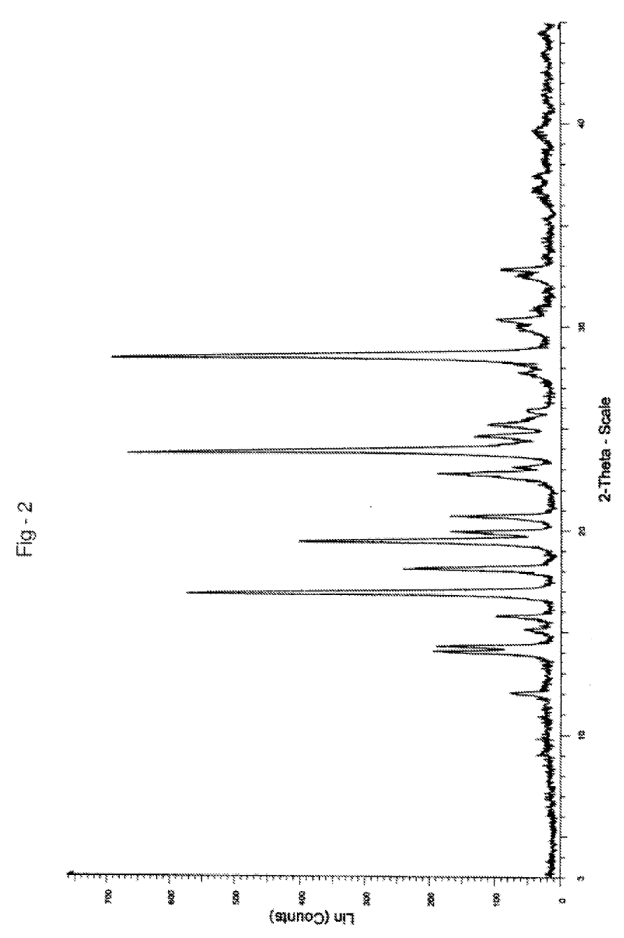

The crystalline 1-[2-(2,4-difluorophenyl)-oxiranyl methyl]-1H-1,2,4-triazole of Formula I is characterized by its XRPD pattern substantially in accordance with the pattern of FIG. 2. It is also characterized by an XRPD pattern having significant peaks at about 23.9, 16.9, 28.7, 19.5, and 14.0±0.2 degrees 2θ. It is also characterized by the additional XRPD peaks at about 19.9, 14.2, 20.7, 24.6, and 22.8±0.2 degrees 2θ.

In an embodiment, the process for the preparation of the crystalline form of 1-[2-(2,4-difluorophenyl)-oxiranyl methyl]-1H-1,2,4-triazole of Formula I comprises the steps of:

a) Reacting 2,4-difluoro-2-(1H-1,2,4-triazol-1-yl)acetophenone of Formula III with trimethyl sulphoxonium iodide in the presence of a phase transfer catalyst and a base in a suitable solvent.

b) Isolation of crystalline form of 1-[2-(2,4-difluorophenyl)-oxiranyl methyl]-1H-1,2,4-triazole of Formula I from the reaction mass of step a).

Step a) involves reacting 2,4-difluoro-2-(1H-1,2,4-triazol-1-yl)acetophenone of Formula III with trimethyl sulphoxonium iodide, in the presence of a phase transfer catalyst, and a base in a suitable solvent;

Suitable phase transfer catalysts which can be used include, but are not limited to cetyl trimethyl ammonium bromide, tetrabutyl ammonium bromide, tetrapropyl ammonium bromide, tri butyl benzyl ammonium chloride, tetra ethyl ammonium bromide, tetra butyl ammonium hydrogen sulphate, benzyl trimethyl ammonium chloride, ethyl triphenyl phosophonium bromide, benzyl triethyl ammonium chloride, and the like.

Suitable bases which can be used include, but are not limited to organic bases such as triethylamine, trimethyl amine, di-isopropylethylamine and the like; inorganic bases such as sodium carbonate, sodium bicarbonate, potassium carbonate, potassium bicarbonate, and the like.

Suitable solvents which can be used for the reaction include, but are not limited to alcoholic solvents such as methanol, ethanol, isopropyl alcohol, n-propanol, and the like; halogenated solvents such as dichloromethane, 1,2-dichloroethane, chloroform, carbon tetrachloride and the like; ketonic solvents such as acetone, ethyl methyl ketone, methyl isobutyl ketone and the like; esters such as ethyl acetate, n-propyl acetate, n-butyl acetate, t-butyl acetate and the like; ether solvents such as diethyl ether, dimethylether, diisopropylether, methyl tertiary-butyl ether, tetrahydrofuran, 1,4-dioxane and the like; hydrocarbon solvents such as toluene, xylene, n-heptane, cyclohexane, n-hexane and the like; nitrile solvents such as acetonitrile, propionitrile and the like; dimethylsulfoxide (DMSO), N,N-dimethylformamide (DMF), N,N-dimethylacetamide and the like; or mixtures thereof or their combination with water in various proportions without limitation.

Step b) involves Isolation of crystalline form of 1-[2-(2,4-difluorophenyl)-oxiranyl methyl]-1H-1,2,4-triazole of Formula I from the reaction mass of step a).

Isolation of the crystalline intermediate 1-[2-(2,4-difluorophenyl)-oxiranyl methyl]-1H-1,2,4-triazole of Formula I can be done by concentrating the organic layer obtained after completion of the reaction to a minimum volume and cooling it to isolate crystalline 1-[2-(2,4-difluorophenyl)-oxiranyl methyl]-1H-1,2,4-triazole of Formula I.

Concentration may be carried out suitably using evaporation, atmospheric distillation or distillation under vacuum.

Distillation of the organic layer may be conducted under vacuum, such as below about 100 mm Hg to below about 600 mm Hg, at elevated temperatures such as about 20° C. to about 70° C. Any temperature and vacuum conditions can be used as long as there is no increase in the impurity levels of the product.

Concentration of the solution can be carried out to an extent where the volume of the reaction mass is equal to 0.5 times to 1.0 time to the weight of the starting material taken.

After the completion of the concentration, the reaction mass may be maintained further at temperatures lower than the concentration temperatures such as for example below about 10° C. to about 25° C., for a period of time as required for a more complete isolation of the product. The exact cooling temperature and time required for complete crystallization can be readily determined by a person skilled in the art and will also depend on parameters such as concentration and temperature of the solution or slurry.

Isolation of the separated solid can be conducted by conventional techniques such as filtering, decanting, centrifuging and the like, or by filtering under an inert atmosphere using gases such as for example nitrogen and the like.

Isolation of the crystals can be conducted at lower temperatures of 5 to 10° C. Higher or lower temperatures are acceptable as long as there is no further generation of impurities.

The isolated solid may optionally be washed with a less polar organic solvent, at a temperature of below about 20 to 25° C. The choice of temperature will of course depend on the solubility of the crystals at that temperature. A temperature at which the solubility is the lowest is preferred to optimize yields.

The less polar organic solvents which can be used for washing the solid include, but are not limited to hydrocarbon solvents such as toluene, xylene, n-heptane, cyclohexane, n-hexane and the like or mixtures thereof.

The wet cake may optionally be further dried. Drying can be suitably carried out in a tray dryer, vacuum oven, air oven, or using a fluidized bed drier, spin flash dryer, flash dryer and the like. The drying can be carried out at temperatures of about 35° C. to about 70° C. The drying can be carried out for any desired time periods, times about 1 to 20 hours.

In a process of the present invention, the formation of a solid intermediate product of Formula I provides a purification method not possible with the prior process in which 2,4-difluoro-2-(1H-1,2,4-trazol-1-yl)acetophenone of Formula III is reacted with trimethyl sulphoxonium iodide, cetrimide, and sodium hydroxide in a solvent to form a crude form of 1-[2-(2,4-difluorophenyl)-oxiranyl methyl]-1H-1,2,4-triazole of Formula I. Thus, relatively impure starting materials of Formula III can be employed in the present process and purification effected by isolation of the intermediate of Formula I prior to proceeding to the next stage.

The crystalline product of Formula I is obtained with an improved yield with greater throughput than with the prior art process. In the prior art process method, when the total organic layer is distilled off completely at higher temperatures of greater than 70° C., the reaction mixture produces relatively high levels of decomposition products and color bodies, which makes isolation of the desired compound of Formula I difficult and renders an impure product.

Another aspect of the invention involves a process for preparing fluconazole of Formula II using the crystalline intermediate of Formula I.

In an embodiment, the process for the preparation of fluconazole of Formula II from the crystalline intermediate of Formula I comprises the reaction of the crystalline intermediate of Formula I with 1,2,4-triazole and a base in a suitable solvent.

Suitable bases which can be sued for the reaction include, but are not limited to: alkali metal alkoxides such as sodium methoxide, sodium ethoxide or its solution in alcohol, potassium methoxide, potassium ethoxide or its solution in alcohol, sodium tertiary butoxide, potassium tertiary butoxide, sodium secondary butoxide, sodium tertiary butoxide, and the like; alkali metal hydroxides comprising sodium hydroxide, potassium hydroxide, and lithium hydroxide; alkali metal carbonates such as sodium carbonate, sodium bicarbonate, potassium carbonate, potassium bicarbonate, lithium carbonate, and the like; alkali metal hydrides comprising sodium hydride, and the like; or mixtures thereof.

Suitable solvents which can be used for the reaction include, but are not limited to alcoholic solvents such as methanol, ethanol, isopropyl alcohol, n-propanol, and the like; halogenated solvents such as dichloromethane, 1,2-dichloroethane, chloroform, carbon tetrachloride and the like; ketonic solvents such as acetone, ethyl methyl ketone, methyl isobutyl ketone and the like; esters such as ethyl acetate, n-propyl acetate, n-butyl acetate, t-butyl acetate and the like; ether solvents such as diethyl ether, dimethylether, diisopropylether, methyl tertiary-butyl ether, tetrahydrofuran, 1,4-dioxane and the like; hydrocarbon solvents such as toluene, xylene, n-heptane, cyclohexane, n-hexane and the like; nitrile solvents such as acetonitrile, propionitrile and the like; dimethylsulfoxide (DMSO), N,N-dimethylformamide (DMF), N,N-dimethylacetamide and the like; or mixtures thereof or their combinations with water in various proportions without limitation.

After the completion of the reaction, the product may be recrystallized or slurried in a suitable solvent for purification.

Suitable solvents which can be used for recrystallization include, but are not limited to: water; alcoholic solvents such as methanol, ethanol, isopropyl alcohol, n-propanol, and the like; halogenated solvents such as dichloromethane, 1,2-dichloroethane, chloroform, carbon tetrachloride and the like; ketonic solvents such as acetone, ethylmethyl ketone, methyl isobutyl ketone and the like; esters such as ethyl acetate, n-propyl acetate, n-butyl acetate, t-butyl acetate and the like; ether solvents such as diethyl ether, dimethylether, di-isopropylether, methyltertiarybutyl ether, tetrahydrofuran, 1,4-dioxane and the like; hydrocarbon solvents such as toluene, xylene, n-heptane, cyclohexane, n-hexane and the like; nitrile solvents such as acetonitrile, propionitrile and the like; dimethylsulfoxide (DMSO), N, N-dimethylformamide (DMF), N,N-dimethylacetamide and the like; or mixtures thereof or their combinations with water in various proportions without limitation.

Additionally, the organic layer containing the product may be washed with an aqueous acidic solution for further purification.

Suitable acids which can be used for the preparation of the aqueous acidic solution include, but are not limited to inorganic acids such as hydrochloric, hydrobromic, hydroiodic, sulphuric, and phosphoric as well as organic acids such as para-toluenesulphonic, methanesulphonic, oxalic, carbonic, succinic, citric, benzoic, and acetic acid and related inorganic and organic acids.

These and other aspects and embodiments of this invention are described in further detail by the examples below, which examples are not intended to limit the scope of the appended claims in any manner.

### EXAMPLE 1

### Preparation of Crystalline 1-[2-(2,4-difluorophenyl)-oxiranyl methyl]-1H-1,2,4-triazole (Formula I)

150 g of 2,4-difluoro-2-(1H-1,2,4-trazol-1-yl) acetophenone of Formula III and 2250 ml of toluene were taken into a round bottom flask and stirred. 155 g of trimethyl sulfoxonium iodide and 4.5 g of cetyl trimethyl ammonium bromide were added to the above reaction mass. The reaction mass was cooled to 20° C. and a solution of 60 g of caustic lye flakes in 300 ml of deionized water was added to the above reaction mass at 20 to 22° C. After the completion of the addition, the reaction mass was heated to 47° C. and maintained for 9 hours. The reaction mass was then cooled to 28° C. and filtered under vacuum. The aqueous layer was separated from the filtrate and extracted with 150 ml of toluene. The combined toluene layer was given carbon treatment with 5 g of carbon. The toluene layer was distilled under a vacuum of 300 mm/Hg at 44° C. till the volume of the remaining mass was 80 ml. The remaining reaction mass was then cooled to −8° C. the reaction mass was maintained at −8° C. under stirring for 1 hour. The reaction mass was then filtered and washed with 37.5 ml of chilled toluene followed by 100 ml of n-hexane. The compound was dried at 30° C. for four hours to yield 106 g of the title compound in the form of a light brown colored crystalline powder.

Purity by HPLC: 97.7%.

FIG. 2 represents the X-ray powder diffraction pattern for the compound obtained.

### EXAMPLE 2

### Preparation of 2-(2,4-difluorophenyl)-1,3-bis-(1H-1,2,4-triazol-1-yl)-1 propanol (Formula II)

702 ml of isopropanol and 50 g of 1-[2-(2,4-difluorophenyl)-oxiranyl methyl]-1H-1,2,4-triazole obtained above was taken into a round bottom flask. 22.5 g of 1,2,4-triazole and 63.7 g of potassium carbonate were added to the above reaction mass under stirring. The reaction mass was heated to 75° C. The reaction mass was maintained at 75° C. for 16 hours. Reaction completion was checked using thin layer chromatography. After completion of the reaction, the reaction mass was cooled to 30° C. The reaction mass was then filtered and the filter bed was washed with 70 ml of isopropanol in two equal lots. The filtrate was taken into a separate round bottom flask and distilled at 65° C. To the residue obtained 140.5 ml of deionized water was added. The reaction mass was maintained at 66° C. for 30 minutes. The reaction mass was then cooled to 10° C. The separated solid was filtered and washed with 14 ml of deionized water. The wet material was taken into a separate round bottom flask and 267 ml of deionized water was added to it. The reaction mass was heated to 98° C. 2.25 g of carbon was charged into the reaction mass and maintained at 98° C. for 30 minutes. The reaction mass was filtered and the carbon bed was washed with 32.5 ml of deionized water. The filtrate was cooled to 12° C. and maintained for 45 minutes. The separated solid was filtered and washed with 32.5 ml of deionized water. The wet material was taken into another round bottom flask and 1220 ml of chloroform was added to it. The reaction mass was checked for clear dissolution. 1030 ml of water, 51.5 g of citric acid, and 103 g of sodium chloride were taken into a separate round bottom flask and stirred for 5 minutes. The chloroform layer obtained above was washed with the above solution in 3 equal lots. The organic layer was then subjected to distillation under a vacuum of 300 mm Hg at 45° C. 103 ml of water was charged to the residue obtained and heating given to 96° C. 1.2 g of carbon was added to the reaction mass and maintained at 96° C. for 30 minutes. The reaction mass was then filtered under hot conditions and the carbon bed was washed with 32.5 ml of deionized water. The filtrate was cooled to 10° C. and maintained for 1 hour. The separated compound was filtered and washed with 26 ml of chilled deionized water. The wet compound was dried in oven at 65° C. for 2 hours to yield 34 g of the title compound.

Purity by HPLC: 99.98%.

What is claimed is:

1. Crystalline 1-[2-(2,4-difluorophenyl)-oxiranyl methyl]-1H-1,2,4-triazole.

2. The crystalline 1-[2-(2,4-difluorophenyl)-oxiranyl methyl]-1H-1,2,4-triazole of claim 1, having an X-ray diffraction pattern obtained with Cu Kα-1 radiation substantially in accordance with FIG. 2.

3. The crystalline 1-[2-(2,4-difluorophenyl)-oxiranyl methyl]-1H-1,2,4-triazole of claim 1, having an X-ray diffraction pattern obtained with Cu Kα-1 radiation with peaks at about 23.9, 16.9, 28.7, 19.5, and 14.0±0.2 degrees 2θ.

4. The crystalline 1-[2-(2,4-difluorophenyl)-oxiranyl methyl]-1H-1,2,4-triazole of claim 3, having an X-ray diffraction pattern obtained with Cu Kα-1 radiation with additional peaks at about 19.9, 14.2, 20.7, 24.6, and 22.8±0.2 degrees 2θ.

5. A process for preparing crystalline 1-[2-(2,4-difluorophenyl)-oxiranyl methyl]-1H-1,2,4-triazole, comprising reacting 2,4-difluoro-2-(1H-1,2,4-triazol-1-yl)acetophenone of Formula III with trimethyl sulphoxonium iodide in the presence of a phase transfer catalyst and a base.

6. The process of claim 5, further comprising crystallizing 1-[2-(2,4-difluorophenyl)-oxiranyl methyl]-1H-1,2,4-triazole.

7. The process of claim 5, wherein a phase transfer catalyst comprises at least one of cetyl trimethyl ammonium bromide, tetrabutyl ammonium bromide, tetrapropyl ammonium bromide, tri butyl benzyl ammonium chloride, tetra ethyl ammonium bromide, tetra butyl ammonium hydrogen sulphate, benzyl trimethyl ammonium chloride, ethyl triphenyl phosophonium bromide, and benzyl triethyl ammonium chloride.

8. The process of claim 5, wherein a phase transfer catalyst comprises cetyl trimethyl ammonium bromide.

9. A process for preparing fluconazole, comprising:
a) reacting 2,4-difluoro-2-(1H-1,2,4-triazol-1-yl)acetophenone of Formula III with trimethyl sulphoxonium iodide in the presence of a phase transfer catalyst and a base;
b) crystallizing and isolating 1-[2-(2,4-difluorophenyl)-oxiranyl methyl]-1H-1,2,4-triazole formed in a); and
c) reacting 1-[2-(2,4-difluorophenyl)-oxiranyl methyl]-1H-1,2,4-triazole from b) with 1,2,4-triazole and a base.
